(19) United States
(12) Patent Application Publication
(10) Pub. No.: US 2020/0155837 A1
(43) Pub. Date: May 21, 2020

(54) SKIN CARE DEVICE

(71) Applicant: GCS CO., LTD., Seongnam-si, Gyeonggi-do (KR)

(72) Inventor: Chang Sik KIM, Suwon-si, Gyeonggi-do (KR)

(73) Assignee: GCS CO., LTD., Seongnam-si, Gyeonggi-do (KR)

(21) Appl. No.: 16/619,462

(22) PCT Filed: Mar. 8, 2019
(86) PCT No.: PCT/KR2019/002768

§ 371 Date: Dec. 5, 2019

(30) Foreign Application Priority Data
Mar. 13, 2018 (KR) 10-2018-0029009

(51) Int. Cl. A61N 1/32; A61N 1/40; A61N 1/36

(52) U.S. Cl. CPC A61N 1/327; A61N 1/36031; A61N 1/328; A61N 1/40

(57) ABSTRACT

A skin care device includes: a plasma pulse generator for generating a plasma pulse; an electroporation pulse generator for generating an electroporation pulse; a pulse output unit for measuring a voltage value of a skin of a user by coming into contact with the skin of the user, and outputting at least one of the plasma pulse or the electroporation pulse to the skin of the user; and a controller for analyzing a skin condition of the user using the skin voltage value, and controlling the generation and output of the plasma pulse and electroporation pulse according to the analyzed skin condition.

### TECHNICAL FIELD

Embodiments of the present invention relate to a skin care device.

### BACKGROUND ART

In general, as people age, the skin becomes wrinkled and saggy due to loss in elasticity. In order to address this issue, people use a variety of cosmetics for maintenance of skin elasticity. However, there is a problem that the cosmetics fail to be sufficiently absorbed into the skin because the skin functions to protect the human body from external stimuli or materials.

In this regard, development has been made of a variety of skin care devices, such as a device for removing waste materials on the skin using current and devices for taking care of the skin using vibration, low frequency, galvanism, heat, ozone, etc. In addition, recently, techniques using an electroporation technique incorporated in beauty devices have been developed. Electroporation is one method of introducing genes into cells, in which DNA is introduced into cells by applying an electric field (or high voltage) to the cells to increase permeability of cell membranes. However, a conventional electroporation device or ion introducer simply outputs a frequency for absorption promotion without considering the dryness level of the skin of a user. In addition, conventional high-voltage electroporation uses a high voltage of several hundred or several thousand volts with a narrow pulse width. However, this method instantaneously induces electroporation between cell membranes of the skin, but has a problem that it is difficult to continuously use and it may be applied to a portion of the face, causing pain.

Further, because the conventional high-voltage electroporation applies a high voltage to the skin, it has a problem of increasing stimulus to the skin when used for a long time. For this reason, the conventional high-voltage electroporation should be used only for a short time. However, this condition may not be strictly kept according to users.

### DISCLOSURE

### Technical Problem

Embodiments of the present invention provide a skin care device which is capable of performing an electroporation function and emitting plasma.

### Technical Solution

In accordance with an embodiment of the present invention, a skin care device includes a plasma pulse generator for generating a plasma pulse, an electroporation pulse generator for generating an electroporation pulse, a pulse output unit for measuring a voltage value of a skin of a user by coming into contact with the skin of the user, and outputting at least one of the plasma pulse or the electroporation pulse to the skin of the user, and a controller for analyzing a skin condition of the user using the skin voltage value, and controlling the generation and output of the plasma pulse and electroporation pulse according to the analyzed skin condition.

### Advantageous Effects

According to embodiments of the present invention, it is possible to effectively care for a user's skin through an electroporation function and plasma emission.

Further, according to the embodiments of the present invention, plasma is generated and output in an arc manner, thereby making it possible to minimize the amount of ozone to be generated and solve safety problems resulting from generation of ozone.

According to the embodiments of the present invention, plasma is generated using a high voltage within the range of an intermediate frequency, thereby making it possible to enhance absorptivity of a wrinkle improvement ingredient such as an oligonucleotide.

### DESCRIPTION OF DRAWINGS

FIG. 1 is a block diagram illustrating a skin care device according to one embodiment of the present invention.

FIG. 2 is a block diagram illustrating a skin care device according to another embodiment of the present invention.

FIG. 3 is a view illustrating the configuration of the skin care device according to the other embodiment of the present invention.

FIGS. 4 and 5 are views illustrating voltages output from the skin care device according to the one embodiment of the present invention.

### BEST MODE

In accordance with an embodiment of the present invention, a skin care device includes a plasma pulse generator for generating a plasma pulse, an electroporation pulse generator for generating an electroporation pulse, a pulse output unit for measuring a voltage value of a skin of a user by coming into contact with the skin of the user, and outputting at least one of the plasma pulse or the electroporation pulse to the skin of the user, and a controller for analyzing a skin condition of the user using the skin voltage value, and controlling the generation and output of the plasma pulse and electroporation pulse according to the analyzed skin condition.

The skin care device may further include a relay unit including one or more relays connected between the electroporation pulse generator and the pulse output unit, and the controller may control the output of the electroporation pulse by turning the one or more relays on or off.

The electroporation pulse generator may include a positive pulse generator for providing a positive electroporation pulse to the pulse output unit, and a negative pulse generator for providing a negative electroporation pulse to the pulse output unit, and the relay unit may include a first relay connected between the positive pulse generator and one terminal of the pulse output unit, and a second relay connected between the negative pulse generator and the other terminal of the pulse output unit.

The controller may control the first relay and the second relay, respectively, such that at least one of the positive electroporation pulse or the negative electroporation pulse is output.

The plasma pulse generator may include a transformer, and may generate the plasma pulse by applying a voltage to the transformer.

The transformer may have an output stage connected to the electroporation pulse generator and the pulse output unit.

### [Mode for Invention]

Reference will now be made in detail to the exemplary embodiments of the present invention, examples of which are illustrated in the accompanying drawings. The following detailed description is provided to aid in comprehensive understanding of a method, a device and/or a system described in this specification. However, this is merely illustrative, and the present invention is not limited thereto.

In the following description of embodiments of the present invention, a detailed description of known functions and configurations incorporated herein will be omitted for the purpose of clarity and for brevity. The terms to be described below are terms defined in consideration of functions in the present invention, and may be changed according to the intention or custom of a user or operator. Therefore, the definition will be formed based on the entire contents of the present specification. The terms in the detailed description are merely used for description of the embodiments of the present invention, and the present invention should not be limited thereto. The singular expressions in the present specification include the plural meanings unless clearly specified otherwise in context. It will be further understood that the terms “comprises” or “comprising” when used in this specification specify the presence of stated features, integers, steps, operations, elements, or combinations thereof, but do not preclude the presence or addition of one or more other features, integers, steps, operations, elements, or combinations thereof.

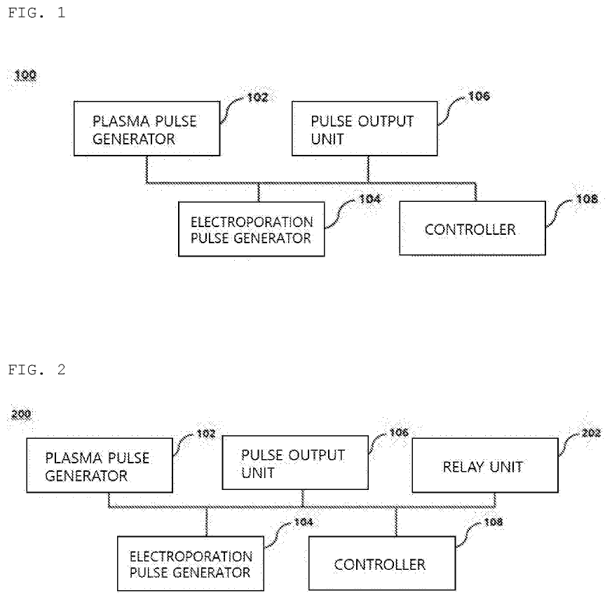

FIG. 1 is a block diagram illustrating a skin care device 100 according to one embodiment of the present invention.

Referring to FIG. 1, the skin care device 100 according to the one embodiment of the present invention outputs at least one of a plasma pulse or an electroporation pulse to a user's skin to care for the user's skin, and includes a plasma pulse generator 102, an electroporation pulse generator 104, a pulse output unit 106, and a controller 108.

The plasma pulse generator 102 generates a plasma pulse.

In detail, the plasma pulse generator 102 may generate a plasma pulse in response to a plasma pulse generation request from the controller 108. Here, the plasma pulse may be, for example, a pulse of plasma emitted to regenerate the user's skin. In addition, the plasma pulse generator 102 may generate a plasma pulse of a high voltage (for example, 1 kVpp) of a specific frequency range (for example, 30 kHz to 100 kHz).

The electroporation pulse generator 104 generates an electroporation pulse.

In detail, the electroporation pulse generator 104 may generate an electroporation pulse in response to an electroporation pulse generation request from the controller 108. Here, electroporation serves to form a passage on the user's skin to promote absorptivity of, for example, a drug, an active ingredient, etc., and the electroporation pulse may be a pulse for performing this function.

For example, the electroporation pulse generator 104 may include a triangle wave generator for generating a triangle wave to generate a pulse voltage, and a comparator for comparing the level of the triangle wave with that of a direct current (DC) voltage. With this configuration, with reference to a DC voltage determining upper and lower limit levels, the electroporation pulse generator 104 may generate a pulse voltage having a pulse width defined by a section at which the generated triangle wave and the DC voltage cross each other. Here, because the pulse width becomes wider or narrower as the DC voltage increases or decreases in level, the controller 108 may determine the frequency of the electroporation pulse by adjusting the level of the DC voltage. In addition, the electroporation pulse generator 104 may generate electroporation pulses of various shapes by performing a charge pumping operation under the control of the controller 108. It should be noted that this is merely one example for description of the present invention and the present invention is not limited thereto, and the electroporation pulse generator 104 may generate a pulse in the same manner as a conventional electroporation voltage generation method.

The pulse output unit 106 outputs at least one of a plasma pulse or an electroporation pulse to the skin of the user by coming into contact with the skin of the user.

In detail, the pulse output unit 106 may be electrically connected to the plasma pulse generator 102 to receive a plasma pulse from the plasma pulse generator 102 and output the received plasma pulse to the skin of the user. In addition, the pulse output unit 106 may be electrically connected to the electroporation pulse generator 104 to receive an electroporation pulse from the electroporation pulse generator 104 and output the received electroporation pulse to the skin of the user. For example, the pulse output unit 106 may be connected in parallel to the plasma pulse generator 102 and the electroporation pulse generator 104. It should be noted that this is merely one example for description of the present invention, and the scheme of connection between the pulse output unit 106 and the plasma pulse generator 102 and electroporation pulse generator 104 is not limited thereto.

The pulse output unit 106 measures the voltage value of the skin of the user by coming into contact with the skin of the user.

In detail, the pulse output unit 106 may measure the voltage value of the skin of the user under the condition of coming into contact with the skin of the user, and provide the measured voltage value to the controller 108. Here, the skin voltage value measured by the pulse output unit 106 may be, for example, an analog value.

The controller 108 analyzes the condition of the skin of the user using the skin voltage value provided from the pulse output unit 106. For example, in the case where the skin voltage value provided from the pulse output unit 106 is an analog value, the controller 108 may convert the skin voltage value into a digital value using an analog-to-digital converter (ADC) and then analyze the skin condition of the user based on the digital value.

In detail, the controller 108 may store a plurality of skin voltage value ranges and information about skin condition by skin voltage value ranges, determine a skin voltage value converted into a digital value belongs to which one of the skin voltage value ranges, and analyze the skin condition of the user based on a result of the determination. Here, the controller 108 may store a plurality of skin voltage value ranges and information about skin condition by skin voltage value ranges in a database (for example, a read only memory (ROM), a random access memory (RAM) or a memory card) existing inside or outside the skin care device 100.

The controller 108 controls the generation and output of the plasma pulse and electroporation pulse according to the skin condition of the user.

In detail, the controller 108 may store output pulse information about each skin condition and determine a pulse to be output through the pulse output unit 106, according to the skin condition of the user. Here, the controller 108 may store the output pulse information about each skin condition in the above database. The output pulse information about each skin condition may include, for example, the type of a pulse to be output according to each skin condition, the amplitude of the pulse to be output, and a pulse output period.

For example, in the case where the skin condition of the user is determined to be a first condition and output pulse information about the first skin condition is stored in the controller 108 as outputting only the electroporation pulse, the controller 108 may control the electroporation pulse generator 104 and the pulse output unit 106 such that only the electroporation pulse is generated and output. At this time, the controller 108 may provide a voltage to the electroporation pulse generator 104 such that the electroporation pulse generator 104 generates the electroporation pulse.

For another example, in the case where the skin condition of the user is determined to be a second condition and output pulse information about the second skin condition is stored in the controller 108 as outputting the plasma pulse and the electroporation pulse in the ratio of 5 to 5, the controller 108 may control the plasma pulse generator 102, the electroporation pulse generator 104 and the pulse output unit 106 such that the plasma pulse and the electroporation pulse are generated and output in the ratio of 5 to 5. At this time, the controller 108 may apply voltages respectively to the plasma pulse generator 102 and the electroporation pulse generator 104 such that the plasma pulse generator 102 and the electroporation pulse generator 104 generate the plasma pulse and the electroporation pulse, respectively.

For another example, in the case where the skin condition of the user is determined to be a third condition and output pulse information about the third skin condition is stored in the controller 108 as selectively outputting any one of the plasma pulse and the electroporation pulse, the controller 108 may selectively output any one of the plasma pulse and the electroporation pulse. At this time, for example, the controller 108 may select any one of the two pulses arbitrarily or under the control of the user of the skin care device 100 and output the selected pulse through the pulse output unit 106.

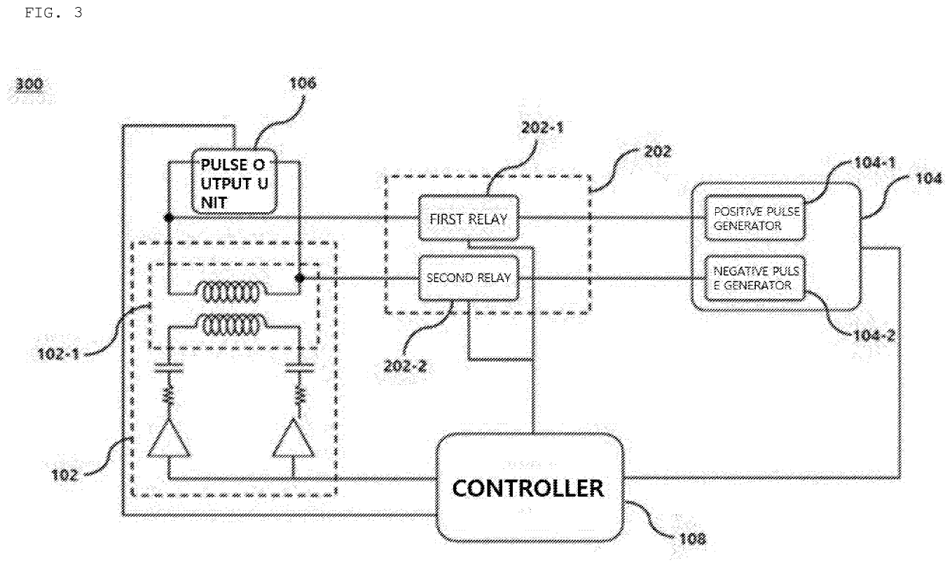

FIG. 2 is a block diagram illustrating a skin care device 200 according to another embodiment of the present invention.

Referring to FIG. 2, the skin care device 200 according to the other embodiment of the present invention may further include a relay unit 202.

The relay unit 202 may include one or more relays connected between the electroporation pulse generator 104 and the pulse output unit 106. Here, the controller 108 may control the output of the electroporation pulse by turning on or off the one or more relays included in the relay unit 202.

In detail, the relay unit 202 may be electrically connected between the electroporation pulse generator 104 and the pulse output unit 106. The controller 108 may open or close the electrical connection between the electroporation pulse generator 104 and the pulse output unit 106 by turning on or off the one or more relays included in the relay unit 202.

On the other hand, although not shown in FIG. 2, the skin care device 200 according to the other embodiment of the present invention may include a condition output unit (not shown).

The condition output unit may provide information associated with skin care to the user. In detail, the condition output unit may provide information associated with skin care to the user through output devices, for example, a light emitting device, a motor, a display device, and an audio output device. Here, the skin care-associated information may include, for example, the skin condition of the user, and information about a pulse output through the pulse output unit 106 (for example, information about the type, amplitude and frequency of the pulse).

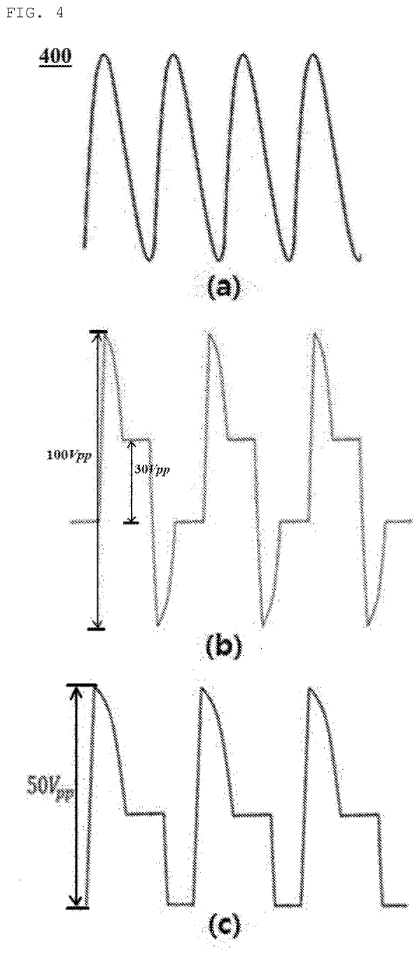

FIG. 3 is a view 300 illustrating the configuration of the skin care device 200 according to the other embodiment of the present invention.

Referring to FIG. 3, the electroporation pulse generator 104 may include a positive pulse generator 104-1 and a negative pulse generator 104-2. Here, the positive pulse generator 104-1 may provide a positive (+) electroporation pulse to the pulse output unit 106, and the negative pulse generator 104-2 may provide a negative (−) electroporation pulse to the pulse output unit 106. On the other hand, the positive electroporation pulse may mean, for example, a positive (+) part of the electroporation pulse, and the negative electroporation pulse may mean, for example, a negative (−) part of the electroporation pulse.

The relay unit 202 may include a first relay 202-1 and a second relay 202-2. Here, the first relay 202-1 may be connected between the positive pulse generator 104-1 and one terminal of the pulse output unit 106, and the second relay 202-2 may be connected between the negative pulse generator 104-2 and the other terminal of the pulse output unit 106.

The controller 108 may control the first relay 202-1 and the second relay 202-2, respectively, such that at least one of the positive electroporation pulse or the negative electroporation pulse is output. For example, the controller 108 may turn on the first relay 202-1 to electrically connect the positive pulse generator 104-1 to the pulse output unit 106, so as to output the positive pulse generated by the positive pulse generator 104-1 through the pulse output unit 106. For another example, the controller 108 may turn off the second relay 202-2 to open the electrical connection between the negative pulse generator 104-2 and the pulse output unit 106, so that the negative pulse generated by the negative pulse generator 104-2 is not transferred to the pulse output unit 106.

The plasma pulse generator 102 may include a transformer 102-1. In detail, because a high voltage is required to generate plasma in an arc manner, the plasma pulse generator 102 may include the transformer 102-1 for generating the high voltage. Here, the turn ratio of the transformer 102-1 may be selected within the range of, for example, 500 to 3000.

In addition, the plasma pulse generator 102 may include, for example, a switching device such as a field effect transistor (FET), a resistor, and a capacitor. In detail, the plasma pulse generator 102 may include switching devices, resistors, and capacitors connected in series, respectively, to both ends of a primary coil of the transformer 102-1. Here, the resistors and the capacitors may be included to stabilize a voltage applied to the primary coil of the transformer 102-1.

The plasma pulse generator 102 may generate a plasma pulse by applying a voltage to the transformer 102-1. In detail, the plasma pulse generator 102 may apply a voltage to the primary coil of the transformer 102-1 such that a plasma pulse is generated by an insulator or dielectric formed between the primary coil of the transformer 102-1 and a secondary coil thereof. In addition, the generated plasma pulse may be output in an arc manner through the pulse output unit 106 connected to the secondary coil of the transformer 102-1. In this case, both ends of the secondary coil of the transformer 102-1 may be connected respectively to both terminals of the pulse output unit 106.

The transformer 102-1 may have an output stage connected to the electroporation pulse generator 104 and the pulse output unit 106. In detail, the output stage of the transformer 102-1 may be connected in parallel to the relay unit 202 connected to the electroporation pulse generator 104, and the pulse output unit 106. On the other hand, FIG. is merely one example for description of the present invention, and the configuration of the skin care device 200 is not limited thereto.

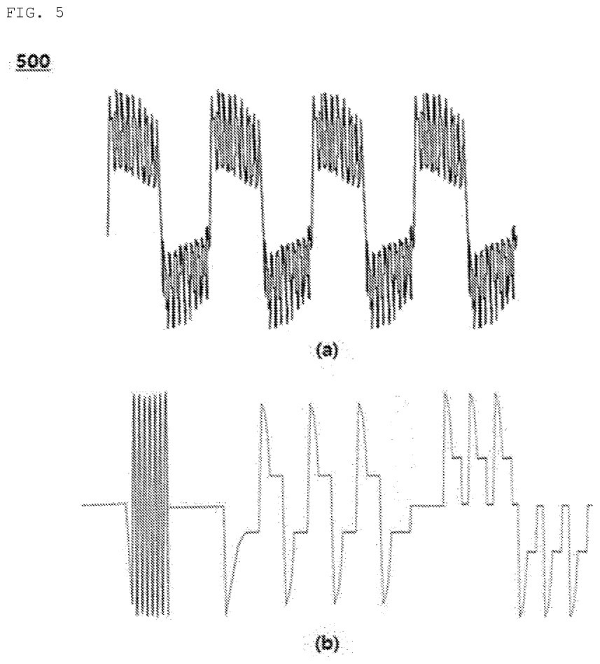

FIGS. 4 and 5 are views 400 and 500 illustrating voltages output from the skin care device 200 according to the one embodiment of the present invention.

In detail, FIG. 4(a) shows a waveform of a plasma pulse which is generated by the plasma pulse generator 102 and output through the pulse output unit 106. A voltage induced in the secondary coil of the transformer 102-1 may have a sinusoidal waveform as shown in this drawing. Here, the controller 108 may control the plasma pulse generator 102 such that the plasma pulse has, for example, a frequency of 30 kHz to 100 kHz and an output voltage of 1 kVpp or more. In addition, in order to enhance skin absorptivity of a wrinkle improvement ingredient such as an oligonucleotide, the controller 108 may control the plasma pulse generator 102 such that the plasma pulse has a frequency of 40 kHz.

FIGS. 4(b) and 4(c) show waveforms of an electroporation pulse which is generated by the electroporation pulse generator 104 and output through the pulse output unit 106. In detail, FIG. 4(b) shows a waveform of an alternating current (AC) voltage which is output through the pulse output unit 106 when the controller 108 controls the relay unit 202, and FIG. 4(c) shows a waveform of a direct current (DC) voltage which is output through the pulse output unit 106 when the controller 108 controls the relay unit 202.

FIGS. 5(a) and 5(b) show waveforms of a plasma pulse and an electroporation pulse which are generated respectively by the plasma pulse generator 102 and the electroporation pulse generator 104 and together output through the pulse output unit 106. In detail, FIG. 5(a) shows waveforms of the plasma pulse and electroporation pulse when output simultaneously, and FIG. 5(b) shows waveforms of the plasma pulse and electroporation pulse when output sequentially.

Although the exemplary embodiments of the present invention have been disclosed for illustrative purposes, those skilled in the art will appreciate that various modifications, additions and substitutions are possible, without departing from the scope and spirit of the invention. Accordingly, the scope of the present invention should be determined by reasonable interpretation of the accompanying claims, and all equivalent modifications made without departing from the scope of the present invention should be understood as being included in the following claims.

1. A skin care device comprising:
a plasma pulse generator for generating a plasma pulse;
an electroporation pulse generator for generating an electroporation pulse;
a pulse output unit for measuring a voltage value of a skin of a user by coming into contact with the skin of the user, and outputting at least one of the plasma pulse or the electroporation pulse to the skin of the user; and
a controller for analyzing a skin condition of the user using the skin voltage value, and controlling the generation and output of the plasma pulse and electroporation pulse according to the analyzed skin condition.

2. The skin care device according to claim 1, further comprising a relay unit comprising one or more relays connected between the electroporation pulse generator and the pulse output unit,
wherein the controller controls the output of the electroporation pulse by turning the one or more relays on or off.

3. The skin care device according to claim 2, wherein the electroporation pulse generator comprises a positive pulse generator for providing a positive electroporation pulse to the pulse output unit, and a negative pulse generator for providing a negative electroporation pulse to the pulse output unit,
wherein the relay unit comprises a first relay connected between the positive pulse generator and one terminal of the pulse output unit, and a second relay connected between the negative pulse generator and the other terminal of the pulse output unit.

4. The skin care device according to claim 3, wherein the controller controls the first relay and the second relay, respectively, such that at least one of the positive electroporation pulse or the negative electroporation pulse is output.

5. The skin care device according to claim 1, wherein the plasma pulse generator comprises a transformer, the plasma pulse generator generating the plasma pulse by applying a voltage to the transformer.

6. The skin care device according to claim 5, wherein the transformer has an output stage connected to the electroporation pulse generator and the pulse output unit.
